(19) United States
(12) Patent Application Publication
(10) Pub. No.: US 2004/0073150 A1
(43) Pub. Date: Apr. 15, 2004

(54) Ambulatory trans-lumbar traction system

(76) Inventor: Thomas C. Roballey, Huntington, CT (US)

Correspondence Address: MELVIN K. SILVERMAN, SUITE 440, 4901 NORTH FEDERAL HIGHWAY, Fort Lauderdale, FL, 33308, US

(21) Appl. No.: 10/270,060

(22) Filed: Oct. 15, 2002

(51) Int. Cl. A61F 5/00

(52) U.S. Cl. 602/36

(57) ABSTRACT

An ambulatory trans-lumbar traction system includes a flexible mid-torso brace proportioned for encirclement of the body of the user around the rib cage starting at a level of about the T8 vertebrate, and centered at the T12 vertebrae, the brace having an enlarged dorsal width of about four vertebrae and a frontal width of about one-half of the dorsal width, and includes a tension-variable securement of ends of the brace. The system also includes a flexible pelvic brace proportioned for encirclement about the body, the brace centered at about the level of the L4 vertebrate, and having a peripheral geometry substantially similar to that of the torso brace, the pelvic brace including a tension variable securement of the ends of the brace. Further included are at least two vertically symmetrically disposed cooperative, rigid cylinder and piston pairs vertically secured between each of the dorsal and front sides of the torso and pelvic braces. The system also includes an assembly for independent selectable inflation of each cylinder of each cylinder-piston pair to a desired pressure within a range of about 75 to about 200 pounds per square inch.

### BACKGROUND OF THE INVENTION

1. Field of Invention

This invention relates to devices to aid in the healing of mid-and lower back injuries, and to support the lower back to prevent the re-occurrence of injuries.

2. Prior Art

Spinal injuries, particularly those of the lower back, have been a problem throughout human history. This is due to the relative ease with which injuries to the spine and supporting muscles can occur and, as well, the debilitating effect of even a slight injury. Aggravating the situation is that the most frequently prescribed treatment (short of surgical intervention), for a spine or back-related injury and, particularly, an injury of the lower back, is the cessation or curtailment of almost all physical activities likely to give rise to tortional or compressional stresses upon the spinal cord and, particularly, upon the discs and cartilages between the vertebrate thereof. See FIGS. 1A to 1D. Thereby, due to the pervasive effect of the anatomy of the back upon all but the most sedentary and isolated physical activities, a patient may become substantially immobilized to provide to the injured area sufficient opportunity to heal.

In the above context, the term “injury” may be construed to include not only actually compression and tortional injuries to the various structures of the back but, as well, general strains to large muscle groups interacting with the various anatomical regions which surround the spinal cord.

The human spinal column is a major component of a skeletal system of thirty-three bones comprising seven cervical (C1 to C7), twelve thoracic (T1 to T12), and five lumbar vertebrae (L1 to L5) with the latter merging into five-fused sacral (S1 to S5), and four-fused coccyx, vertebrae. (See FIG. 1D). The twenty-four individual vertebrae have various bony projections with one projection directed outwardly from the back of the spine, this known as the spinous process which can be felt along the back as hard knobs. The individual vertebrae are connected and supported by various cartilages, muscle and ligaments which allow flexibility for bending and twisting of the torso. Between each vertebra is an intervertebral disc which functions to cushion and separate each vertebra, helping to prevent compression of the peripheral nerve roots branching off from the spinal cord and housed within the spinal column. The intervertebral disc also allows for motion between the vertebrae. Displacement of one or more of the individual vertebrae from its normal position, or improper motion, via direct compression or through swelling, can create pressure against the spinal nerves, typically resulting in pain, frequently severe in intensity. Such displacement is often the result of unequal tension of the muscles supporting the spinal column (see FIG. 1E), causing one or more of the individual vertebrae to be pulled out of alignment with the rest. This unequal tension can be caused from a variety of factors, these including over-exertion, posture, uneven muscular stress, emotional tension and direct physical trauma.

A great majority of back pain experienced by the general public occurs in the lower portion of the back generally referred to as the lumbar region, or spinal segments L-3 through S-1 specifically. (See FIG. 1) Once the affected vertebrae of this region are re-aligned, and allowed to move, the pressure exerted against the nerve can be alleviated, resulting in reduction or elimination of pain.

The lumbar spine can be injured in essentially three ways, namely, excessive compression or excessive torsion or a lack of normal intervertebral motion. See FIG. 1B. If the former occurs, the most common result is a damaged or fractured vertebral endplate. The preferable remedy for such injury is rest, or a near complete avoidance of any physical activity likely to place stress on the spinal column. Corrective surgery is rarely required, as is typical of most back injuries. However, if excessive torsion or twisting occurs, the most common result is a damaged intervertebral disc. Relatively minor torsional injuries, if not allowed to heal, may result in a significantly weakened disc, which is then susceptible to more serious injury. The lumbar spine is generally more susceptible to injury by torsion than by compression. Continued twisting toward an injured side may aggregate the injury and significantly interfere with the healing process. If the vertebral segments are not moving freely, as they were designed, osteoarthritis (degenerative arthritis) begins which weakens the structures and my result in swelling, bulging discs, nerve root compression and pain.

Because the human spine is the essential load bearing component in the human skeleton, any injury to the spine almost inevitably causes at least some discomfort, immobility or pain. After an injury to the spine has occurred, the spine must be given an opportunity to heal itself. Spinal motion in the direction of the injury must be avoided if the injury is not to be aggravated, and given an opportunity to heal. If such opportunity is not provided, an injury may never heal, or become severely aggravated, causing increasing discomfort, physical deterioration, and incapacitation to the affected individual. However, because the spine is in constant everyday use, it is continuously subject to stresses which may interfere with the healing process.

A multitude of devices have been designed to prevent the occurrence of lower back injuries, such as support belts and braces for workers engaged in repetitive lifting activities, or for the general populace during occasional lifting or athletic endeavors. Also, there are devices designed to be worn during everyday activities by individuals already exhibiting lower back injury symptoms. These devices, which can often be similar in design to the preventative devices, allow the wearer to engage in activities while, in theory, still removing sufficient stress from the lower back to permit some healing of existing injuries, obviating the need for complete inactivity during the healing process. Lastly, there is a class of devices, generally of a far more complex scope mechanically, that are designed to provide active or positive therapeutic benefit, typically in a clinical setting to the injured user. Mechanical traction devices are exemplary of such devices. The present invention relates to such a system, however to one which does not require use in a clinical setting.

In the third category of back devices described above, there are a number of braces and other such devices which are designed to provide active therapeutic benefit and to promote healing of the injured area. Generally, these devices can range from full scale clinical appliances in the form of tables, chairs or other like structures, to belts and slings designed to be used in conjunction with large appliances. In theory, these devices function by suspending the weight of the affected patient in a manner that almost totally removes all gravitational stresses from the affected area of the spine. Traction devices are part of an aggressive, non-surgical, or post-surgical regimen designed to keep the spine free from torsional and compressive forces, thus allowing the injured area to heal as rapidly and effectively as possible. The major drawback of most tractional therapies is that, due to the complexity of the apparatus and the need for substantial intervention by an appropriately trained health care professional to assure proper therapeutic use and optimal benefit, they are suited only for use in a controlled clinical settings. Such time that a patient spends in a normal traction device must therefore be dedicated time during which the patient is incapable of participating in other activities.

Said third category of back devices is reflected in such specific prior art as U.S. Pat. No. 4,991,572 to Chases which discloses a lumbar harness designed to employ the principles of gravity traction of relieve stresses from the lumbar spinal region to thereby permit efficient healing of the affected area. The Chases device employs air-inflated bladders to increase the comfort of the patient during traction therapy. However, the device of Chases, and other structures of this type, is such that the patient must dedicate time to such therapy and cannot pursue normal daily activities during this time.

Devices which employ air-inflated bladders to effect an ambulatory form of traction therapy include U.S. Pat. No. 5181,904 (1993) to Cook and U.S. Pat. No. 5,704,904 (1998) to Dunfee, also known as the Orthotrac Vest. Dunfee, while sharing a number of the objectives of the instant invention, relates only to an inflatable lumbar traction vest in which circumferential horizontal belts, as well as integral vertical members associated therewith, all comprise bladders which, thereby, are substantially limited in the degree of vertical and radial traction which can be applied to the lumbar and sacral areas. Stated otherwise, the forces which an inflatable vest can impart to the patient is of limited value in stabilizing the spine to preclude aggravation of an existing injury. As such, the positive therapeutic effect which can be derived from such inflatable bladder devices is minimal due to the low inflation pressure of approximately twenty pounds per square inch that can be sustained in such bladders. A much higher pressure is necessary to effect a meaningful therapeutic effect.

Accordingly, some in the art have recognized the importance of use of rigid vertical mechanical elements to apply constant contraction to at least the lumbar area and to provide for re-directed or asymmetrical traction, thereby producing a non-vertical resultant force against given parts of the spine to thereby address, in the context of an ambulatory system, lumbar spine disorders requiring high pressure or asymmetrical traction. Such a system is manufactured in Israel by the Meditrac Corporation and distributed in the United States by Warwick Associates of Tennessee. This track system however consists of rigid, uncomfortable vertical elements which, as a practical matter, cannot be adjusted by the patient in a straightforward and safe fashion. Examples of such purely mechanical ambulatory traction belts may be found in U.S. Pat. No. 3,926,182 (1975) to Stabholz, U.S. Pat. No. 3,960,146 (1976) to Albrect, and U.S. Pat. No. 4,114,611 (1978) to Lyle.

The instant invention addresses the above set forth needs and limitations of the prior art through the suggestion of as ambulatory lumbar traction system which employs means for the provision of necessary air pressure to vary the length of rigid vertical piston members to apply a meaningful level of traction to the user.

### SUMMARY OF THE INVENTION

An ambulatory trans-lumbar traction system includes a flexible mid-torso brace proportioned for encirclement of the body of the around the lower rib cage starting at a level of about the T10 vertebrate, said brace having an enlarged dorsal width of about four vertebrae, centered at the T12 vertebrae, and having a frontal width of about one-half of said dorsal width, said brace including means for selectably tension-variable securement to each other of ends of said flexible brace. The system also includes a flexible pelvic brace proportioned for encirclement about the body, said brace centered at about the level of the L4 vertebrate, said pelvic brace having a peripheral geometry substantially similar to that of said torso brace, said pelvic brace including means for selectable tension variable securement of the ends thereof to each other. Further included are at least two vertically symmetrically disposed cooperative, rigid cylinder and piston pairs vertically secured between each of said dorsal and front sides of said torso and pelvic braces. The system further includes means for independent selectable inflation of each cylinder of each cylinder-piston pair to desired pressure within a range of about 75 to about 200 pounds per square inch. The system yet further includes a planar elastomeric double cross-over lumbar support having a width substantially equal to a least a separation between opposing horizontal edges of said torso and pelvic braces respectively, said support having, upon an inner surface in contact with the body of the user, a planar internal air bladder inclusive of means for the selectable inflation thereof to a desired pressure in a range of about 6 to 25 pounds per square inch. Appropriate check and release valves may be employed in said vertical cylinders to guard against potential overinflation thereof.

It is accordingly an object of the present invention to provide an ambulatory trans-lumbar traction system for providing positive therapeutic benefits to a user thereof and to enhance the healing of injuries of the spinal region

It is another object to provide a traction device of the above type which is adaptable to everyday use by the wearer, applied and managed by the wearer, thereby permitting one to pursue normal daily activities while providing a significant degree of protection to the spine as well as the aforesaid positive active therapeutic benefits.

It is a further object of the invention to provide a device and system of the above type having the advantages of low pressure horizontal force applied against the lumbar region which high-pressure vertical tractional forces are applied to the thoracic lumbar and vertebrae.

It is a yet further object to provide a system of the above type capable of left-to-right, as well as front-to-dorsal, asymmetrical traction.

It is a still further object to provide a lumbar brace suitable for everyday use while providing protection against injury and aggravation of existing injury as well as active therapeutic benefit in the healing of spine-related disorders previously achievable only through the use of a full-scale clinical appliance.

It is a yet further object of the invention to provide an ambulatory device readily usable by sufferers of herniated discs, chronic low back pain and related lumbar spine injuries and disorders, this as an alternative to surgery, body braces, bed traction or the like.

The above and yet other objects and advantages of the present invention will become apparent from the hereinafter set forth Brief Description of the Drawings, Detailed Description of the Invention and Claims appended herewith.

### BRIEF DESCRIPTION OF THE DRAWINGS

FIGS. 1A to 1E are anatomical views of the human spinal column and associated muscles, nerves and tissue.

FIG. 2 is a top plan view of the torso and pelvic brace parts of the present invention inclusive of cylinder and piston means associated therewith.

FIG. 3 is a front perspective operational view showing placement of the torso and pelvic braces upon a user thereof.

FIG. 4 is a front view, similar to that of FIG. 3, however showing the use of an inflatable elastic double crossover lumbar support associated with the present system.

FIG. 5 is a plan view of the elastic lumbar cross-over support and the inflatable surface associated therewith.

FIG. 6 is a rear view of the operational view of the view of FIG. 4.

FIG. 7 is a side view of the operational view of the view of FIG. 4.

FIG. 8 is a schematic view of a switching apparatus for filling of the respective cylinders of the system to respectively different pressures, as may be indicated by given anatomical requirements.

### DETAILED DESCRIPTION OF THE INVENTION

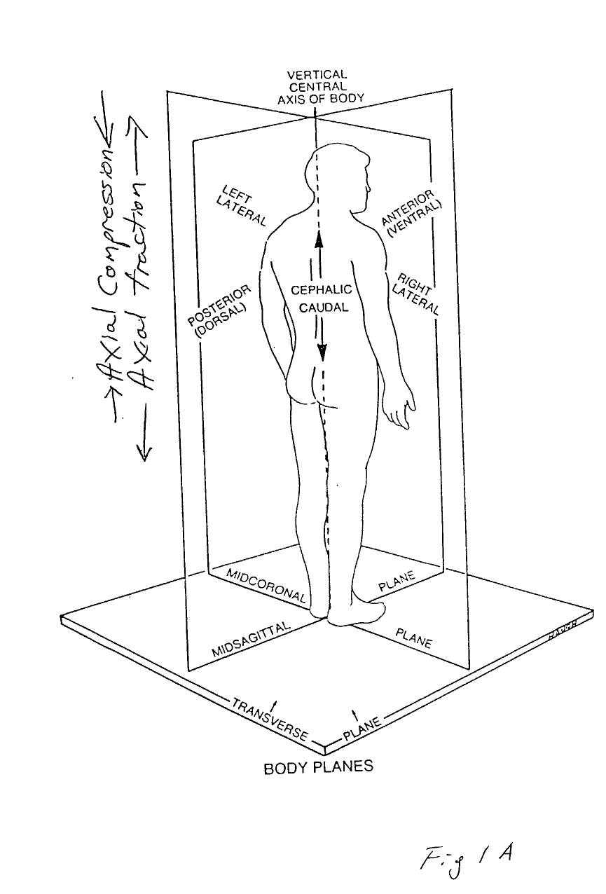

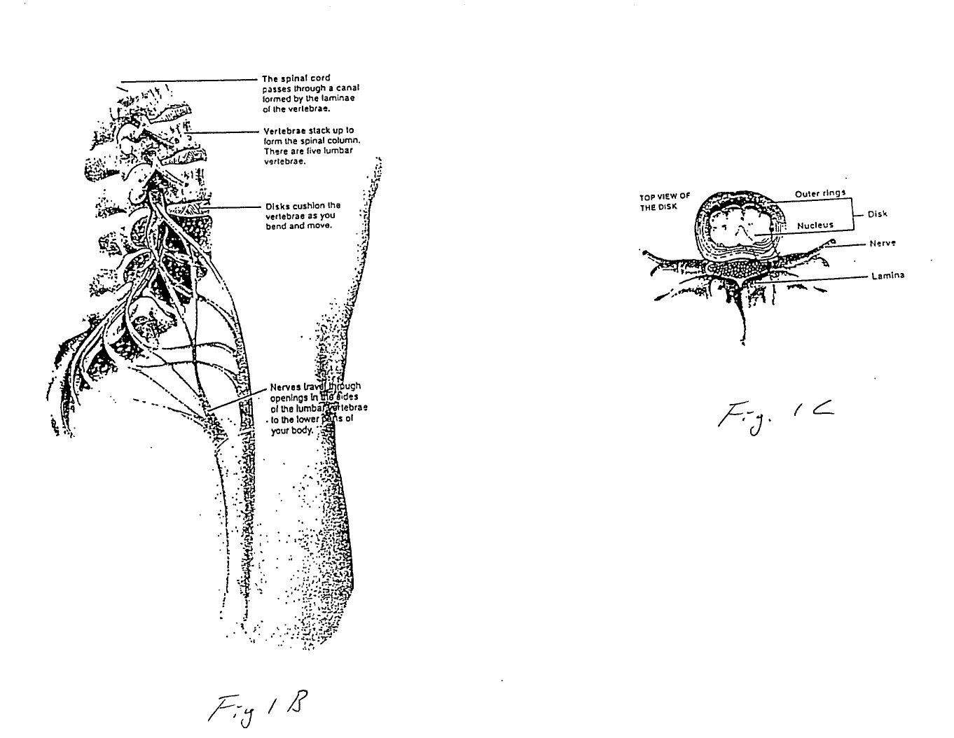

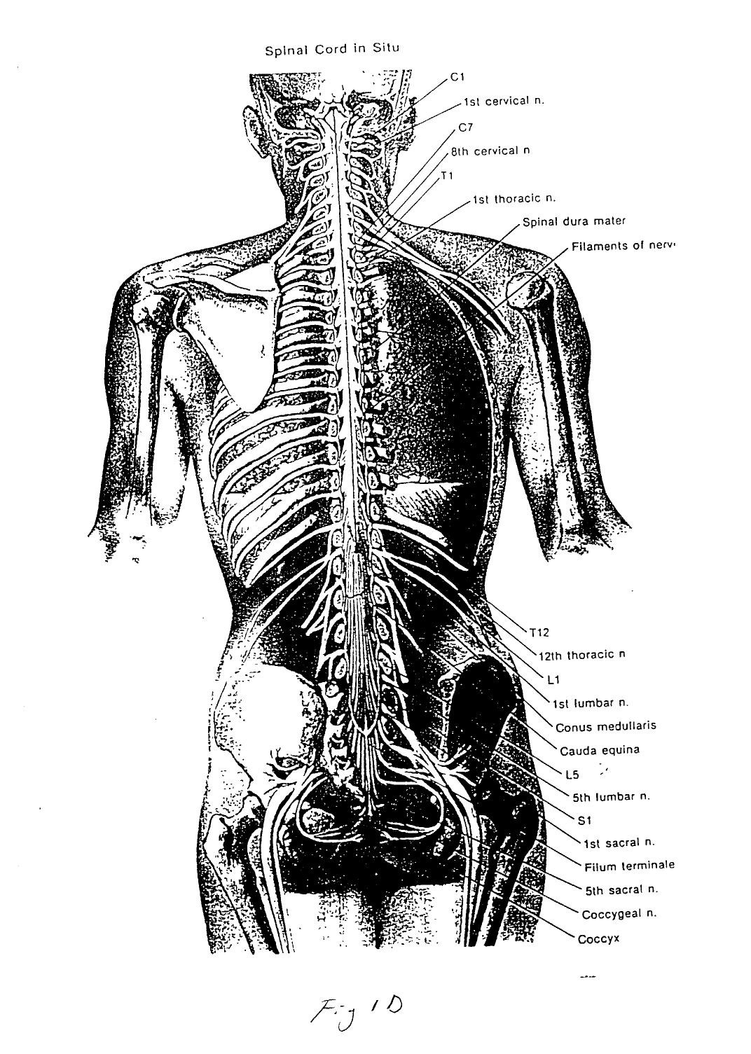

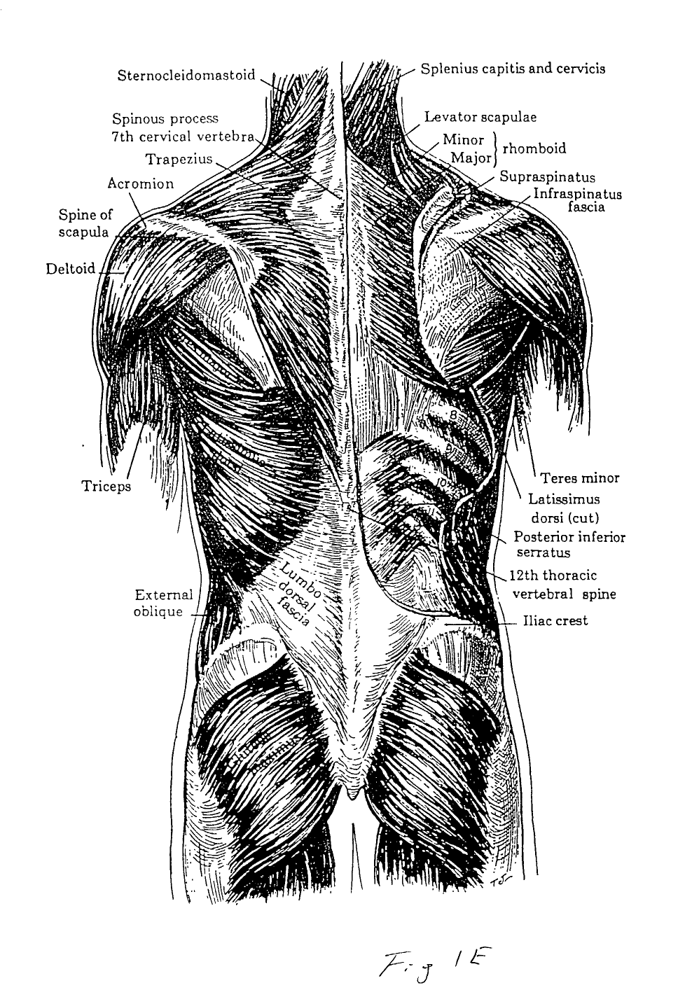

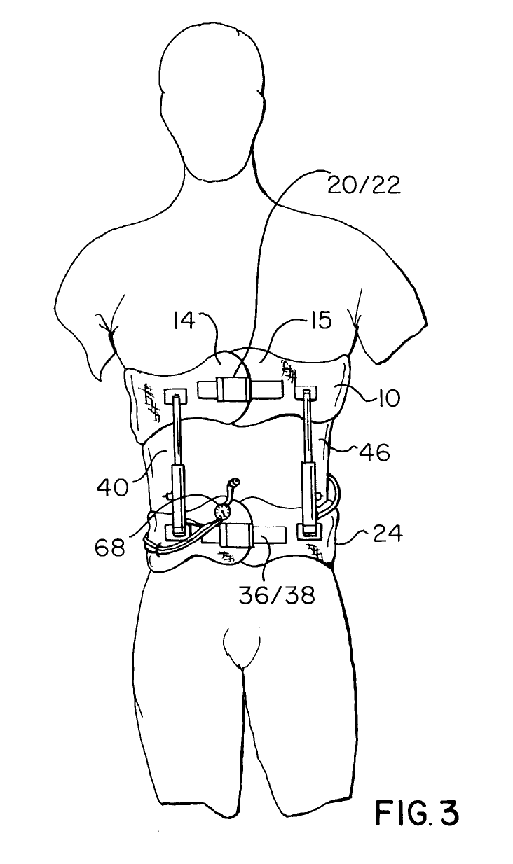

With reference to the views of FIGS. 1D, 2-5, the inventive ambulatory trans-lumbar traction system may be seen to include a flexible mid-torso brace 10 which is proportioned for encirclement of the body of the user beneath and around the lower rib cage and starting at a level of about the T10 vertebrae, centered at the T12 vertebrae, and extending to approximately the level of the L1 vertebrae. See FIG. 1D. As noted, the brace 10 includes an enlarged dorsal portion 12 thereby having a width of 4 to 5 vertebrae and a frontal width 14 which is about one half of the dorsal width of the brace. Integrally depending from ends 16 and 18 of front portions 14 and 15 of brace 10 are means 20 and 22 for the engagement and selectable tension variable securement of ends 14 and 15 toward each other, this in the manner shown in FIG. 3.

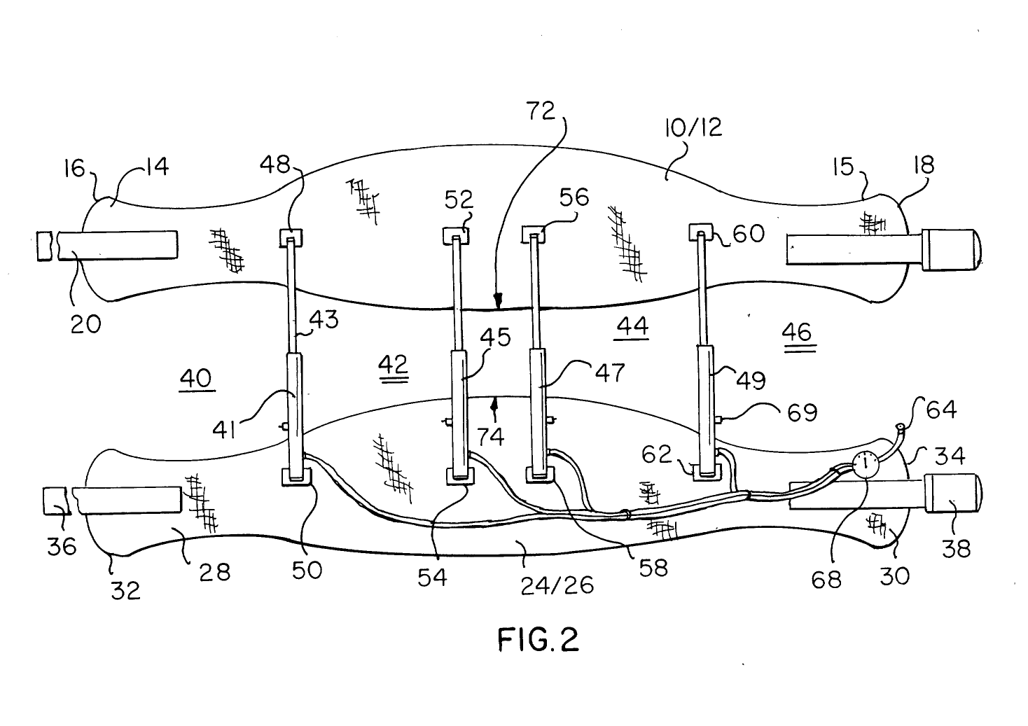

The inventive system further includes a flexible pelvic brace 24 (see FIGS. 2 to 6) which, as in the case of said torso brace 10, includes an enlarged dorsal portion 26 and front portions 28 and 30, each having opposing front ends 32 and 34 which are securable to each other, through the use of engagement and tension variable securement means 36 and 38, to secure pelvic brace 24 at a level centered at approximately the L3 vertebrae. As may be noted, the profile or peripheral geometry of pelvic brace 24 is substantially similar to that of said torso brace 10. Thereby, each provides significant support to the respective areas of the spinal column against which they are applied. Further, the compression achieved thereby also serves as a platform against which tractional forces, as below described, that are associated with the instant system may be imparted to the user.

With further reference to FIGS. 2 to 5, the assembly of FIG. 2 may be seen to further include at least two vertically symmetrically disposed cooperative rigid cylinder and piston pairs 40/42 and 44/46 which are secured to belts 10 and 24 at points 48/50, 52/54 56/58, and 60/62 respectively. As may be noted, each cylinder and piston pair consists of a respective cylinder 41 and piston 43.

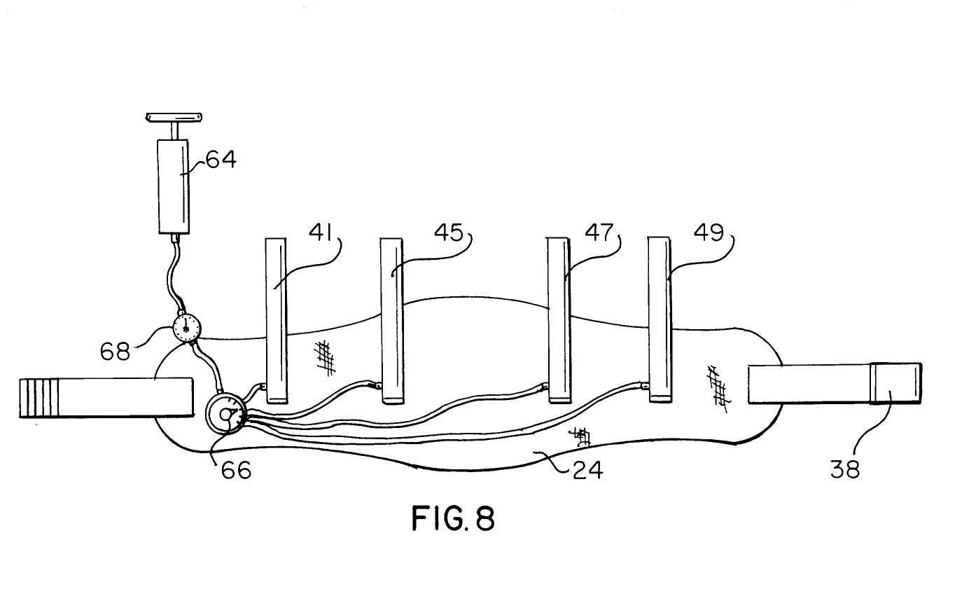

As may be further noted, the system further includes pump means 64 (see also FIG. 8) for independent selectable inflation of each respective cylinder and piston pair to a desired pressure within a range of about 75 to about 200 pounds per square inch. That is, by the use of fluid switch 66 (see FIG. 8) a desired pressure may be separately provided to each piston 41, 45, 47 and 49 of the respective piston and cylinder pairs 40, 42, 44 and 46, this to a level of pressure which is viewable in pressure meter 68.

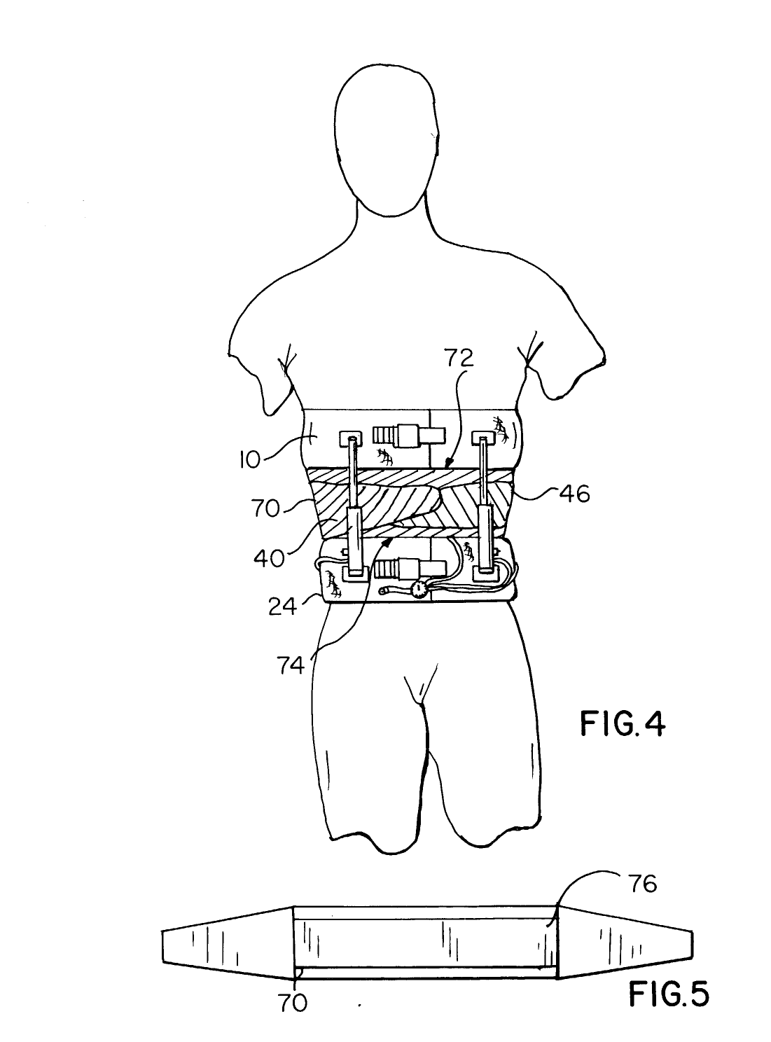

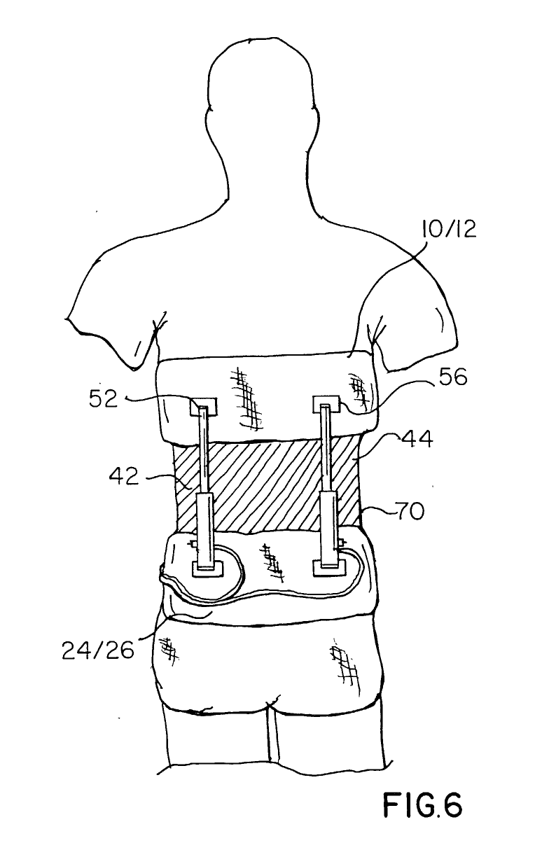

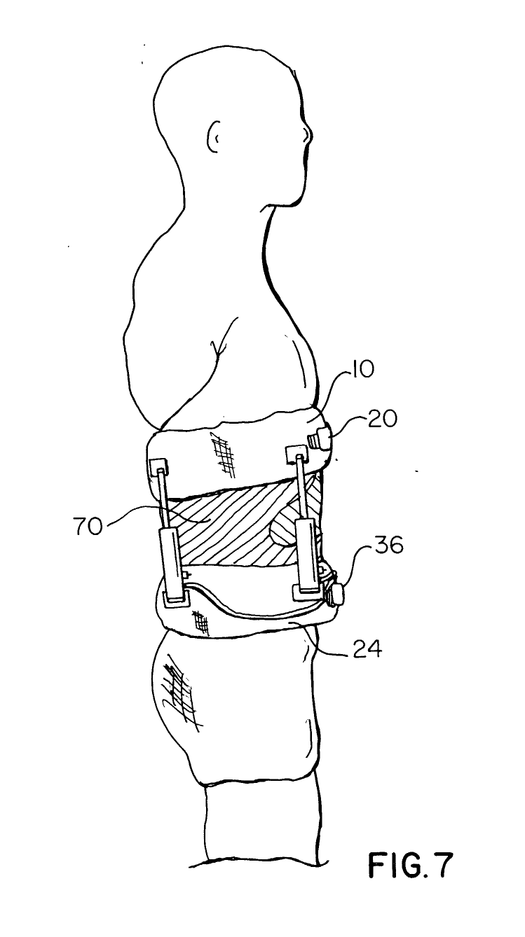

With reference to FIGS. 4 through 6, the inventive system may be seen to yet further include a planar elastomeric double cross-over lumbar support 70 having a width substantially equal to a least separation which exists between opposing horizontal edges 72 and 74 of the respective torso and pelvic braces 10 and 24. As may be more particularly noted with reference to FIG. 5, the lumbar support 70 includes a planar internal air bladder 76 inclusive of means for the selectable inflation thereof to a pressure in a range of about 6 to about 25 pounds per square inch.

With respect to pump means 64 (such as a bicycle pump), it is to be understood that, by virtue of the capability of the cylinders 41-49 to retain a pressure in a range of about 75 to about 200 pounds per square inch, active traction can be applied to the user which, unlike prior art reflected in U.S. Pat. No. 5,704,904 above, enables the application of forces capable of providing positive therapeutic benefit to the spine of patient in the trans lumbar region and which, as well, is entirely ambulatory in nature. Further, through the use of selectably different pressures within respective pistons of the present system, asymmetric left-to-right or front-to-back traction may be provided to accommodate the particular needs of a given patient. Disc herniation/bulge and/or zygoapophyseal joint compression or irritation may respond better to asymmetrical axial traction. For example, a herniated L5 lumbar disc which has protruded posterior and to the right lateral may require more axial traction of cylinder 44 and less at cylinder 40. This would allow for the central disc material (nucleus pulposus) to move toward the midline. By means of ambulatory asymmetrical axial traction one can achieve a negative pressure (vacuum) specifically at the location where the bulging disc material needs to be moved to. Ambulation provides the mechanical means by which this is achieved while asymmetrical traction allows for this motion as well as creating the vacuum.

Circumferential compression of the mid torso also aids in producing a negative pressure within the disc. Circumferential compression in reducing pain will also help with ambulation.

This device by combining asymmetrical axial vertical traction with circumferential compression, provides lumbar/low back decompression, while allowing for ambulation thereby providing a means by which healing will occur. The system is also user friendly in application and operation in a non-clinical setting while providing pain relief and actual therapeutic treatment which can reduce health care costs significantly.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the Claims appended herewith.

Having thus described my invention, what I claim as new, useful, and non-obvious and, accordingly, secure by Letters Patent of the United States is:

1. An ambulatory trans-lumbar traction system comprising:
(a) a flexible mid-torso brace proportioned for encirclement of the body of the user beneath the rib cage thereof, said brace having an enlarged dorsal width of about 5 vertebrae and a frontal width of about one-half thereof, said brace including means for selectable variable-tension securement of front ends thereof to each other.
(b) a flexible pelvic brace proportion for encirclement about the pelvis, including means for the selectable variable-tension securement of front ends thereof to each other;
(c) at least two front-to-dorsal vertically symmetrically disposed co-operative, rigid cylinder and piston pairs, secured between each of said dorsal and front sides of said torso and pelvic braces; and
(d) means for inflation of each cylinder of said cylinder-piston pair to a desired pressure in a range of 75 to about 200 pounds per square inch.

2. The system as recited in claim 1, further comprising:
(f) an inflatable planar elastomeric double crossover lumbar support having a width substantially equal to a separation between opposing horizontal edges of said torso and pelvic braces.

3. The system as recited in claim 1, in which said pelvic brace defines a peripheral geometry substantially similar to that of said torso brace.

4. The system as recited in claim 1, in which said inflation means comprises means for independent selectable inflation of each cylinder-piston pair.

5. The system as recited in claim 2, in which said pelvic brace defines a peripheral geometry substantially similar to that of said torso brace.

6. The system as recited in claim 2, in which said inflation means comprises means for independent selectable inflation of each cylinder-piston pair.

7. The system as recited in claim 3, in which said inflation means comprises means for independent selectable inflation of each cylinder-piston pair.

8. The system as recited in claim 7, further comprising:
(f) an inflatable planar elastomeric double crossover lumbar support having a width substantially equal to a separation between opposing horizontal edges of said torso and pelvic braces.
